(19) United States
(12) Patent Application Publication
(10) Pub. No.: US 2008/0008636 A1
(43) Pub. Date: Jan. 10, 2008

(54) Apparatus for pressure-controlled gas generator

(75) Inventor: Reginald R. Coler, Riverside, CA (US)

Correspondence Address: STETINA BRUNDA GARRED & BRUCKER, 75 ENTERPRISE, SUITE 250, ALISO VIEJO, CA, 92656, US

(73) Assignee: ISCA Technologies

(21) Appl. No.: 11/481,203

(22) Filed: Jul. 5, 2006

(51) Int. Cl. A61L 9/00

(52) U.S. Cl. 422/305

(57) ABSTRACT

According to an aspect of the present invention, there is provided an apparatus for generating a gas. The apparatus includes a first reactant in liquid form and a second reactant capable of generating the gas upon being mixed with the first reactant. The apparatus further includes a feeding container for holding the first reactant, a reaction container for holding the second reactant and the gas generated from mixing the first reactant and the second reactant and an output regulator for releasing the gas from the reaction container to the surrounding. A feeding valve is configured to allow a flow of the first reactant from the feeding container into the reaction container when a feeding container valve pressure is greater than a reaction container valve pressure by a predetermined difference between the feeding container valve pressure and the reaction container valve pressure.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

Carbon dioxide (CO2) is a well-known pest-attractant commonly used in pest traps because it simulates the presence of humans or animals. A trap for haematophagous arthropods is typically baited with a source of CO2, a mechanism for releasing CO2, a termination mechanism for destroying or trapping the pests, and a structure holding all of the mechanisms. In addition, the trap may include visual and thermal profiles to enhance the simulation of an animal host. One common problem for CO2 baited traps is the availability of CO2. Typically, a CO2 baited trap either uses a cylinder of compressed CO2 or an insulated device which holds dry ice (solid CO2) and allows for a gradual release CO2. Users of the CO2 baited traps often find these sources difficult to locate and arrange the logistics for having a constant supply of CO2 from the manufacturers. In addition, there is a health risk working with high-pressure compressed gas cylinder or dry ice tank. They often contain pressures at or around 1000 psi, and if handled improperly can result in serious burns. Less common sources of CO2 are devices that generate CO2 chemically or biologically. These devices, whether they are cultures of yeast in a sugar solution, boluses dropped in water, or sachets of dry chemicals, have up until now been designed to react with all the reagents available without any modulation of the reaction. This lack of control over the reaction rate usually results in either an over-production or an under-production of the CO2 desired for the CO2 baited trap's intended purpose. The inability to intelligently control the rate of CO2 generation according to the need of the CO2 baited trap has inevitably resulted in poor portability of the trap, inefficient use of resources, and/or high manufacture costs.

As such, there is a need in the art for a mechanism to dynamically control CO2 generation according to the requirements of a pest trap.

### BRIEF SUMMARY

According to an aspect of the present invention, there is provided an apparatus for generating a gas. The apparatus includes a first reactant in liquid form, and a second reactant capable of generating the gas upon being mixed with the first reactant. The apparatus further includes a feeding container for holding the first reactant, a reaction container for holding the second reactant and the gas generated from mixing the first reactant and the second reactant. The apparatus further includes a release conduit configured to release an amount of the gas from the reaction container. The apparatus further includes a feeding valve in fluid communication with the feeding container and the reaction container. The feeding valve is configured to allow a flow of the first reactant from the feeding container into the reaction container when a feeding container valve pressure is greater than a reaction container valve pressure by a predetermined difference between the feeding container valve pressure and the reaction container valve pressure.

According to various embodiments, the reaction container may be made of a metal. The reaction container may also be made of a plastic material. The first reactant and the second reactant may be used to produce carbon dioxide. The first reactant may be citric acid and water and the second reactant may be sodium bicarbonate. The first reactant may be water and the second reactant may be citric acid and sodium bicarbonate. The apparatus may utilize a feedback regulator placed between the feeding container and the reaction container to allow a flow of the gas formed in the reaction container from the reaction container into the feeding container. The apparatus may utilize a relief valve placed between the feedback regulator and the reaction container to release excessive gas formed in the reaction container. The feeding container may contain pressurization above atmospheric pressure. The apparatus may utilize a pressure tank placed in fluid communication with the feeding container. The pressure tank is configured to hold a pressure above atmospheric pressure. The apparatus may utilize a pressure regulator placed between the pressure tank and the feeding container to release a substance from the pressure tank into the feeding container. The substance may be a gas. The substance may be air. The substance may also be the first reactant. The reaction container may have an entry port and the feeding container may have an exit port placed at a height greater than the entry port, and the reaction container is connected to the feeding container through the exit port and the entry port. The feeding container may utilize a breather opening placed between the interior wall of the feeding container and the exterior wall of the feeding container to allow air to flow inside the feeding container. The feeding container may be a collapsible bag made of plastic material. An output regulator may be disposed in fluid communication with the release conduit.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic depicting an apparatus with a feedback feeding valve placed between a reaction container and a feeding container capable of incorporating an embodiment of the present invention;

FIG. 2 is a schematic depicting an apparatus with a pressure tank connected to a feeding container according to another embodiment;

FIG. 3 is a schematic depicting an apparatus wherein a feeding container is held at a height above a reaction container according to another embodiment; and

FIG. 4 is a schematic depicting an apparatus wherein a feeding container is a collapsible bag according to another embodiment.

### DETAILED DESCRIPTION

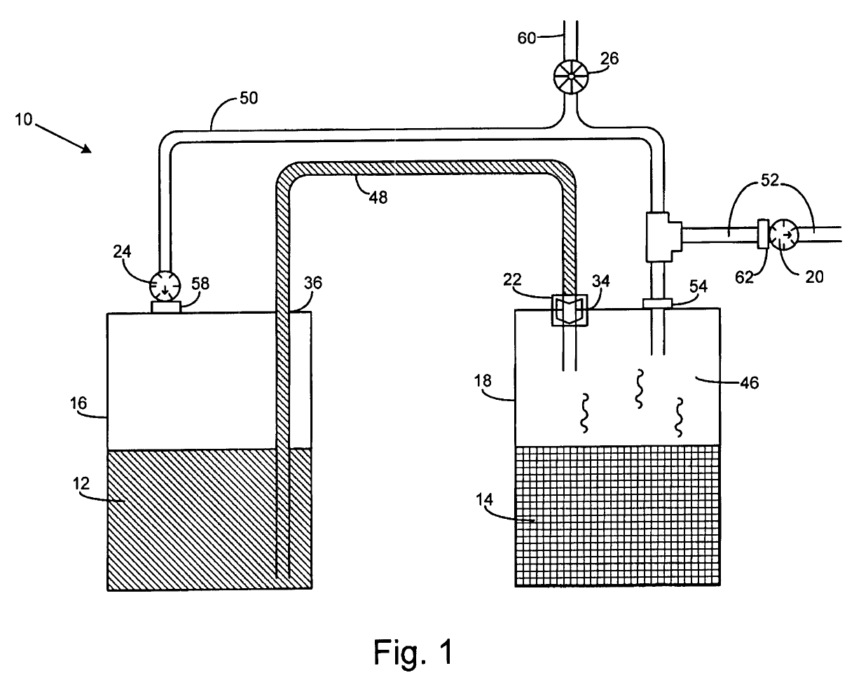

Referring now to FIG. 1, there is depicted an apparatus 10 capable of incorporating an embodiment of the present invention (details of which are discussed below and shown in additional figures). The apparatus 10 generates a product gas 46 which can be used for various purposes. An example of the product gas 46 would be carbon dioxide (CO2) which can be used as a pest attractant. The apparatus 10 in this embodiment includes the feedback mechanism that allows the apparatus 10 to self-regulate the generation of the product gas 46 without requiring an external source to pressurize a feeding container 16.

The apparatus 10 includes a first reactant 12 in liquid form, and a second reactant 14 in solid or liquid form capable of generating a product gas 46 upon being mixed with the first reactant 12. The apparatus 10 further includes the feeding container 16 that holds the first reactant 12, and a reaction container 18 that holds the second reactant 14 and the product gas 46 generated from mixing the first reactant 12 and the second reactant 14. The apparatus 10 further includes a feeding valve 22 in fluid communication with the feeding container 16 and the reaction container 18. As used herein the term “in fluid communication” includes, but is not limited to, any gaseous or liquid communication. As will be discussed in further detail below, the feeding valve 22 controls the flow of the first reactant 12 into the reaction container 18.

In the particular embodiment shown, the apparatus 10 further includes a feeding conduit 48 that connects the feeding container 16 to the reaction container 18. The apparatus 10 further includes an exit port 36 attached to the feeding container 16 and connects the feeding container 16 to the feeding conduit 48, and an entry port 34 attached to the reaction container 18 and connects the reaction container 18 to the feeding conduit 48. The apparatus 10 further includes an output port 54, a release conduit 52, and a feedback conduit 50. The output port 54 is attached to the reaction container 18. The output port 54 is disposed in fluid communication with both the release conduit 52 and the feedback conduit 50. The output port 54 allows flow from the reaction container 18 into the release conduit 52 and the feedback conduit 50. The apparatus 10 further includes a release port 62 and an output regulator 20 attached to the release conduit 52 and the feedback conduit 50 that connects the reaction container 18 to the feeding container 16. The apparatus 10 further includes a feedback regulator 24 attached to the feedback conduit 50 and a feeding port 58 attached to the feeding container 16 and connects the feeding container 16 to the feedback conduit 50. The apparatus 10 further includes a relief conduit 60 that connects to the feedback conduit 50 and a relief valve 26 attached to the relief conduit 60.

The apparatus 10 is characterized by including a feeding container valve pressure and a reaction container valve pressure. As referred to herein the term “feeding container valve pressure” refers to that pressure at the feeding valve 22 which is associated with the feeding container 16 and is otherwise “upstream” from both the feeding valve 22 and the reaction container 18. As referred to herein the term “reaction container valve pressure” refers to that pressure at the feeding valve 22 which is associated with the reaction container 18 and is otherwise “downstream” from the feeding valve 22 and the feeding container 16.

The feeding valve 22 is configured to allow a flow of the first reactant 12 from the feeding container 16 into the reaction container 18 when the feeding container valve pressure is greater than the reaction container valve pressure by a predetermined difference between the feeding container valve pressure and the reaction container valve pressure.

The conduits and the containers can be made of various materials, including metal, plastic, and any other material capable of holding the first reactant 12, the second reactant 14 and the product gas 46 at a predetermined container pressure. In addition, while many of the various components of the apparatus 10 are shown as discrete components that are connected to each other, any number or combination of such components may be integrally formed according to various other embodiments. For examples, the output port 54 may be integrally formed with the reaction container 18, the output port 54 may be integrally formed with the release conduit 52, and the release portion 62 maybe integrally formed with the output regulator 20.

The first reactant 12 can be citric acid and water, and the second reactant 14 can be sodium bicarbonate, and together they can generate CO2 as the product gas 46. The first reactant 12 can be water, and the second reactant 14 can be citric acid mixed with sodium bicarbonate, and together that can also generate CO2 as the product gas 46. The feeding container 16 is normally designed to contain a pressure above atmospheric pressure during the generation process.

Substances in gaseous and liquid form generally flow from a point of higher pressure to a point of lower pressure. Therefore, to set up the apparatus 10 in this embodiment, it is desirable to pressurize the feeding container 16 to contain a higher pressure than a pressure of the reaction container 18. In this regard, one end of the feeding conduit 48 may be submersed in the first reactant 12 in the feeding container 16 to facilitate the flow of the first reactant 12 from the feeding container 16 to the reaction container 18.

To better illustrate the operation of the apparatus 10, sample pressure numbers will be used in the following example. Initially, both the feeding container 16 and the reaction container 18 are at atmospheric pressure. To initialize the apparatus 10, the feeding container 16 may be pressurized by either pumping air through a shrader valve, or by dropping an appropriate amount of the first reactant 12 into the reaction container 18 that will produce the product gas 46 which ultimately pressurizes the apparatus 10 to the appropriate operating pressure. If a shrader valve (not shown) is used to charge the apparatus 10 with air, such a valve must be connected to the feedback conduit 50 and positioned on the feeding container side of a one-way valve (not shown) to prevent flow in a direction of the reaction container 18. In either example, the feeding container pressure may be set to 30 psi as an example. In the process of slowly building up pressure within the feeding container 16, the first reactant 12 will immediately begin to travel through the exit port 36, the feeding conduit 48, the feeding valve 22, and the entry port 34 into the reaction container 18. The first and second reactants 12, 14 will react to ultimately produce the product gas 46. The pressure in the reaction container 18 may thus build to a pressure that will exceed the pressure in the feeding container 16 thereby shutting off the flow at the feeding valve 22 of the first reactant 12 from the feeding container 16.

Assuming the product gas 46 produced from the initial surge of reactant 12 is of a pressure greater than the initial charge pressure of the feeding container 16, the product gas 46 will exit the reaction container 18 via the output port 54 and will travel through the feedback conduit 50 and through the output regulator 24. The feedback regulator may be set for 30 psi, for example. If the pressure of the feeding container 16 has not yet reached 30 psi, additional pressure from either an air pump or un-reacted portions of the first and second reactants 12, 14 will continue to increase until the system reaches the predetermined pressure i.e., 30 psi in this example. Once the feeding container 16 reaches the predetermined operating pressure of 30 psi, actual pressure buildup within the reaction container 18 will likely be higher (e.g., 40-45 psi) because of the continued but slowed reaction taking place within the reaction container 18.

At this juncture, the exhausting of product gas 46 through the release conduit 52 can commence at a desired release rate. The output regulator 20 attached to the release conduit 52 controls the release rate of the product gas 46 by limiting the pressure of product gas 46 that is released. If set at 5 psi, the output regulator 20 will only allow the exiting pressure of the product gas 46 to build-up to 5 psi. The apparatus 10 may be deployed in conjunction with a pest trap. The product gas 46 may be CO2 which may be used as a pest attractant for the pest trap. The CO2 may be leaked into the pest trap at 5 psi as regulated by the output regulator 20.

As the product gas 46 within the reaction container 18 exits the apparatus 10 through the release port 62, the overall pressure within the reaction container 18 will begin to decrease. Once the pressure of the reaction container 18 decreases below 30 psi, the feeding valve 22 will allow the first reactant 12 from the feeding container 16 to flow again into the reaction container 18; within seconds, additional gas built up within reaction container 18 will exceed the pressure of the feeding container 16, and again the flow of reactant 12 will be interrupted. Over time, the pressure of the feeding container 16 will drop as the first reactant 12 flows out of the feeding container 16.

As the apparatus 10 generates more and more product gas 46, some of the product gas 46 flows through the feedback conduit 50 and through the feeding port 58 into the feeding container 16. The feedback flow is designed to pressurize the feeding container 16 by allowing the product gas 46 to enter the feeding container 16 at the predetermined pressure set by the feedback regulator 24 as the first reactant 12 flows out of the feeding container 16. The feedback flow will maintain the pressure inside the feeding container 16. The feedback regulator 24 controls the feedback flow by allowing the product gas 46 to pressurize the feeding container 16 at a predetermined setting. It is noted that the feedback regulator 24 may include a one-way valve to only allow flow into the feeding container 16. When pressure in the feeding container 16 drops below the predetermined setting, the feedback regulator 24 allows flow from the feedback conduit 50 into the feeding container 16.

It is noted that it is undesirable for the feedback regulator 24 to pressurize the feeding container 16 so much that the point of shut off cannot be reached because of the high feeding container pressure. Further, it is noted that it is undesirable for the feedback regulator 24 to pressurize the feeding container 16 too little such that the difference in pressure between the feeding container pressure and the reaction container valve pressure is not enough to activate the feeding valve 22 (i.e., the feeding container valve pressure is not greater than the reaction container valve pressure by the predetermined difference between the feeding container valve pressure and the reaction container valve pressure).

In cases where pressure inside the feedback conduit 50 and the release conduit 52 build up too high for the apparatus 10 to safely hold, the relief valve 26 and the relief conduit 60 will release the product gas 46 to the external environment outside of the apparatus 10 to prevent damage to the apparatus 10.

As mentioned above, it is desirable to initially set up the apparatus 10 to pressurize the feeding container 16 to contain a higher pressure than a pressure of the reaction container 18. However it is contemplated that the feeding container 16 may be pressurized too much. For example, an overly pressurized feeding container 16 may result in a non-stop generation of the product gas 46 because the apparatus 10 cannot reach a point of shut off. The point of shut off occurs when the difference between a feeding container valve pressure and a reaction container valve pressure is equal or less than the predetermined difference between the feeding container valve pressure and the reaction container valve pressure. The point of shut off governs the flow of the first reactant 12 by allowing the apparatus 10 to self-regulate the generation of the product gas 46 once a desired amount of the product gas 46 is produced. Another problem may result where the feeding container 16 is overly pressurized due to all of the first reactant 12 from the feeding container 16 being mixed with the second reactant 14 in the reaction container 18 before any of the product gas 46 is generated. The high feeding container pressure considerably increases the rate of the flow of the first reactant 12 from the feeding container 16 to the reaction container 18. When that rate becomes faster than the rate of generation of the product gas 46 from mixing the first reactant 12 and the second reactant 14, all of the first reactant 12 may flow into the reaction container 16 without stopping because the point of shut off is never reached.

The self-regulating configuration of the apparatus 10 allows it to be made relatively small without compromising the quantity of CO2 over a comparatively longer period of time in comparison to prior art CO2 generators. Other common CO2 sources are either (1) small in size but do not produce sufficient desired quantity of CO2, or (2) do produce sufficient desired quantity of CO2 but occupies a significant amount of space and last only a relatively short period of time. The current invention may facilitate a CO2 source that is relatively small in size and capable of producing sufficient quantities of CO2 over a relatively longer period of time in comparison to the prior art.

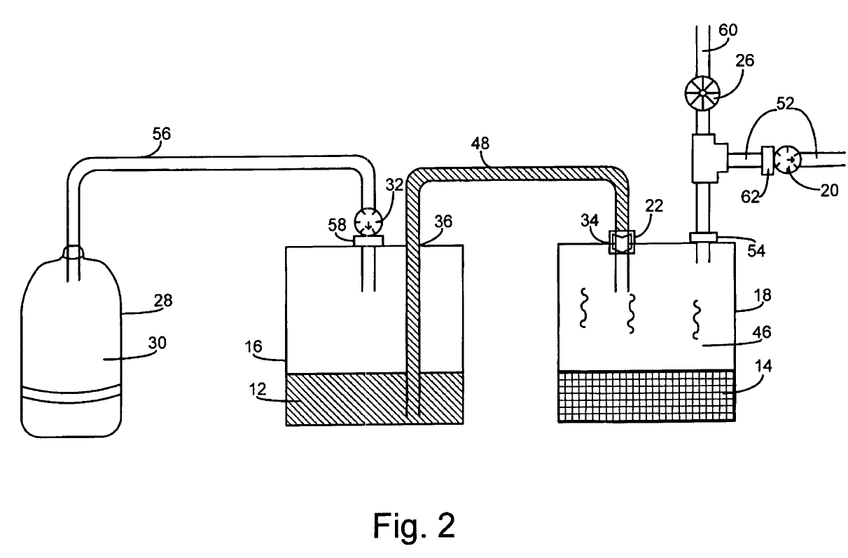

According to another embodiment, referring now to FIG. 2, there is depicted the apparatus 10 using an external source to pressurize the feeding container 16 instead of using the feedback mechanism depicted in the embodiment of FIG. 1. It is noted that like reference numerals are intended to designate like elements as discussed above, however, with differences as discussed and as shown.

In this embodiment, the apparatus 10 includes a pressure tank 28, and a substance 30 contained in the pressure tank 28. The apparatus 10 further includes a supplying conduit 56 that connects to the pressure tank 28 to the feeding container 16. The apparatus 10 further includes a feeding regulator 32 attached to the supplying conduit 56. The apparatus 10 further includes a feeding port 58 attached to the feeding container 16 and connects the supplying conduit 56 to the feeding container 16. It is noted that in this embodiment there is no feedback mechanism as depicted in the embodiment of FIG. 1 to pressurize the feeding container 16. Instead, the feeding container 16 is pressurized by an artificial external source, the pressure tank 28.

As the first reactant 12 flows from the feeding container 16 to the reaction container 18, the pressure of the feeding container 16 drops as the contents of the feeding container 16 is evacuated. When pressure in the feeding container 16 drops below a pre-determined pressure threshold set on the feeding regulator 32, the pressure tank 28 releases the substance 30 into the feeding container 16 through the supplying conduit 56 and the feeding port 58 until the pressure inside the feeding container 16 rises back up to the pre-determined pressure threshold. The feeding regulator 32 controls the flow of the substance 30 by allowing the substance 30 to flow into the feeding container 16 when the pressure inside the feeding container 16 drops below the predetermined pressure threshold set on the feeding regulator 32. It is undesirable for the feeding regulator 32 to pressurize the feeding container 16 too much or too little for the same reasons discussed in the first embodiment depicted in FIG. 1. Because the pressure tank 28 pressurizes the feeding container 16, it is desirable for the pressure tank 28 to contain a higher pressure than the pressure of the feeding container 16. The substance 30 may be air, compressed air, CO2 in liquid form, the first reactant 12, or any combination thereof, for examples. If the substance 30 contains the first reactant 12, the pressure tank 28 also serves as a reservoir of the first reactant 12. Generally the pressure tank 28 will be pressurized to contain a pressure above atmospheric pressure.

In cases where pressure inside the release conduit 52 builds up too high for the release conduit 52 to safely hold, the relief valve 26 and the relief conduit 60 will release the product gas 46 to the external environment outside of the apparatus 10 to prevent damage to the release conduit 52.

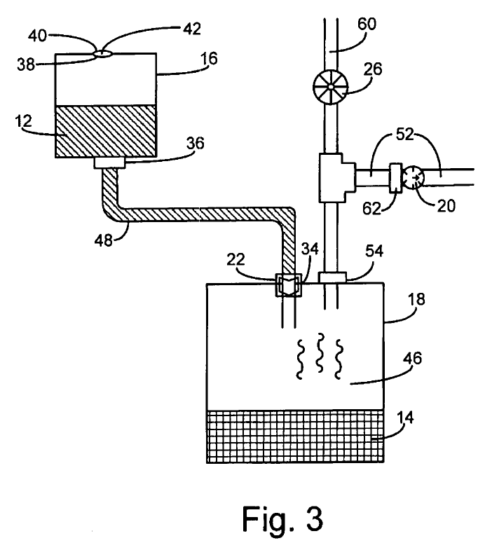

According to another embodiment, referring now to FIG. 3, there is depicted the apparatus 10 using gravity to direct the first reactant 12 to flow from the feeding container 16 into the reaction container 18. In this embodiment, there is no external source that pressurizes the feeding container 16. It is noted that like reference numerals are again intended to designate like elements as discussed above, however, with differences as discussed and as shown.

In this embodiment, the apparatus 10 includes a feeding container interior wall 38, a feeding container exterior wall 40, and a breather opening 42 between the feeding container interior wall 38 and the feeding container exterior wall 40. The breather opening 42 is designed to allow air to flow inside the feeding container 16 as the first reactant 12 flows from the feeding container 16 into the reaction container 18. The inflow of air maintains the pressure of the feeding container 16 at constant atmospheric pressure. The exit port 36 is placed at a height greater than the entry port 34 so that the force of gravity exerts a pressure on the feeding valve 22 to allow the first reactant 12 to flow into the reaction container 18 through the feeding conduit 48 and the entry port 34. The higher the feeding container 16 is positioned above the reaction container 18, the greater the pressure is asserted on the feeding valve 22. Various apparatuses may be used to control the height and the quantity of the first reactant 12 that flows into the reaction container 18. A simple example would be a structure similar to an IV stand used for injecting a hospital patient with liquid medication.

In this embodiment, it is desirable to place the exit port 36 underneath the feeding container 16 to ensure force of gravity is being applied to the feeding valve 22. Once the first reactant 12 mixes with the second reactant 14, the pressure inside the reaction container 18 will build up to the point of shut off and the feeding valve 22 will stop the flow of the first reactant 12. In cases where pressure inside the release conduit 52 builds up too high for the release conduit 52 to safely hold, the relief valve 26 and the relief conduit 60 may be used to release the product gas 46 to the external environment outside of the apparatus 10 to prevent damage to the release conduit 52.

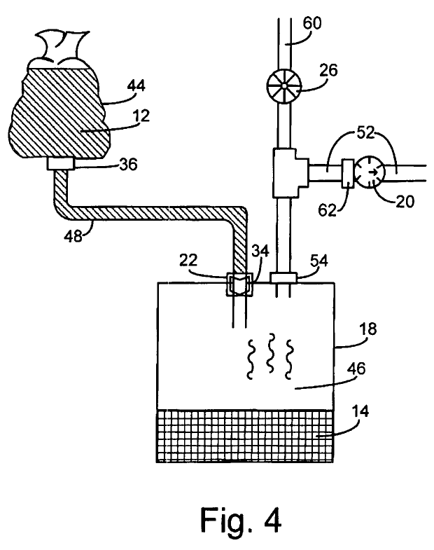

According to another embodiment, referring now to FIG. 4, there is depicted the apparatus 10 which employs a different mechanism to maintain the pressure in the feeding container 16 while still using the force of gravity to direct the first reactant 12 to flow into the reaction container 18. The mechanism involves using a feeding container 16 that is made of flexible material which shrinks as the first reactant 12 flows from the feeding container 16 to the reaction container 18. There is no external source used to pressurize the feeding container 16 as well. It is noted that like reference numerals are again intended to designate like elements as discussed above, however, with differences as discussed and as shown.

In this embodiment, the apparatus 10 includes a collapsible bag 44 as the feeding container 16. The collapsible bag 44 may be made of plastic material. When the first reactant 12 flows out of the collapsible bag 44, the collapsible bag 44 crumples (much like an IV bag) as the contents within the bag is evacuated. As the collapsible bag 44 crumples down to a smaller size, the pressure inside the collapsible bag 44 is kept constant. The advantage of using the collapsible bag 44 is that it can be collapsed to a smaller size when the apparatus 10 is not being used, which allows the apparatus 10 be conveniently stored and/or transported. In cases where pressure inside the release conduit 52 builds up too high for the release conduit 52 to safely hold, the relief valve 26 and the relief conduit 60 may be used to release the product gas 46 to the external environment outside of the apparatus 10 to prevent damage to the release conduit 52.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An apparatus for generating a gas, the apparatus comprising:
a first reactant in liquid form;
a second reactant operative to generate the gas upon being mixed with the first reactant;
a feeding container configured to hold the first reactant;
a reaction container configured to hold the second reactant and the gas generated from mixing the first reactant and the second reactant;
a release conduit configured to release an amount of the gas from the reaction container; and
a feeding valve in fluid communication with the feeding container and the reaction container, the feeding valve being configured to allow a flow of the first reactant from the feeding container into the reaction container when a feeding container valve pressure is greater than a reaction container valve pressure by a predetermined difference between the feeding container valve pressure and the reaction container valve pressure.

2. The apparatus of claim 1, wherein the reaction container comprises a metal.

3. The apparatus of claim 1, wherein the reaction container comprises plastic material.

4. The apparatus of claim 1, wherein the first reactant and the second reactant are operative to produce carbon dioxide.

5. The apparatus of claim 1, wherein the first reactant comprises citric acid and water and the second reactant comprises sodium bicarbonate.

6. The apparatus of claim 1, wherein the first reactant comprises water and the second reactant comprises citric acid and sodium bicarbonate.

7. The apparatus of claim 1 further comprises a feedback regulator in fluid communication with the feeding container and the reaction container, the feedback regulator being configured to allow a flow of the gas formed in the reaction container from the reaction container into the feeding container.

8. The apparatus of claim 7 further comprises a relief valve in fluid communication with the feedback regulator and the reaction container, the relief valve being configured to release the gas formed in the reaction container.

9. The apparatus of claim 8, wherein the feeding container is configured to contain a pressure above atmospheric pressure.

10. The apparatus of claim 9 further comprises a pressure tank configured to contain a pressure above atmospheric pressure, the pressure tank being in fluid communication with the feeding container.

11. The apparatus of claim 10 further comprises a pressure regulator in fluid communication with the pressure tank and the feeding container, the pressure regulator being configured to release a substance from the pressure tank into the feeding container.

12. The apparatus of claim 11, wherein the substance comprises a gas.

13. The apparatus of claim 12, wherein the substance comprises air.

14. The apparatus of claim 11, wherein the substance comprises the first reactant.

15. The apparatus of claim 1, wherein the reaction container includes an entry port, the feeding container including an exit port positionable at a height greater than the entry port, the reaction container and the feeding container being in fluid communication via the exit port and the entry port.

16. The apparatus of claim 15, wherein the feeding container further includes a feeding container interior wall, a feeding container exterior wall, and a breather opening disposed between the feeding container interior wall and the feeding container exterior wall, the breather opening being configured to allow air to flow inside the feeding container.

17. The apparatus of claim 15, wherein the feeding container comprises a collapsible bag.

18. The apparatus of claim 17, wherein the feeding container comprises plastic material.

19. The apparatus of claim 1 further comprising an output regulator disposed in fluid communication with the release conduit.
